(19) United States
(12) Patent Application Publication
(10) Pub. No.: US 2005/0021117 A1
(43) Pub. Date: Jan. 27, 2005

(54) Flexible integrated head-stage for neural interface

(76) Inventors: Jiping He, Tempe, AZ (US); Haixin Zhu, Tempe, AZ (US); Christopher Jennings, Tempe, AZ (US)

Correspondence Address: QUARLES & BRADY LLP, RENAISSANCE ONE, TWO NORTH CENTRAL AVENUE, PHOENIX, AZ, 85004-2391, US

(21) Appl. No.: 10/623,896

(22) Filed: Jul. 21, 2003

(51) Int. Cl. A61N 1/05

(52) U.S. Cl. 607/116

(57) ABSTRACT

An electrode (30) implants into live tissue. The electrode has a first layer with a first silicon portion (50) forming a tip of the electrode and a second benzocyclobutene (BCB) portion (52) disposed adjacent to the first portion. A second BCB layer (56) is disposed over the first layer. A third BCB layer (58) is disposed over the second layer. The first layer further includes a third silicon portion (54) disposed adjacent to the second portion. A head-stage (40) has a connector (38) coupled for receiving the electrical signals from the electrode. A flexible substrate (90) has conductors for transmitting the electrical signals. A stiffener (94) supports a portion of the flexible substrate. An electronic circuit (96) is disposed on the flexible substrate above the stiffener and receives the electrical signals. A connector (12) is supported by the stiffener and coupled to an output of the electronic circuit.

### CLAIM TO BENEFIT OF DOMESTIC PRIORITY

The present non-provisional patent application claims benefit of priority to provisional application Ser. No. 60/397,164, entitled “Flexible Head-stage for Neural Recording in Animal Subjects”, filed on Jul. 19, 2002; and further claims priority to provisional application Ser. No. 60/434,345, entitled “Flexible Integrated Head Stage for Neural Interface”, filed on Dec. 17, 2002; and further claims priority to provisional application Ser. No. 60/434,357, entitled “Implantable Electrode with Flexible Regions to Accommodate Micromovment”, filed on Dec. 17, 2002; and further claims priority to provisional application Ser. No. 60/445,156, entitled “Benzocyclobutene (BCB) as a Biocompatible Material”, filed on Feb. 4, 2003.

### CROSS REFERENCE TO RELATED PATENT APPLICATION(S)

The present patent application is related to copending U.S. patent application Ser. No. ______, Attorney Docket No. 112624.00004, entitled “Electrode for Implant in Live Tissue with Flexible Region to Accommodate Micro-movement”, and filed on Jul. 21, 2003, by Jiping He et al.

### STATEMENT REGARDING FEDERALLY SPONSORED RESEARCH OR DEVELOPMENT

The U.S. Government has a paid-up license in the present invention and the right, in limited circumstances, to require the patent owner to license others on reasonable terms as provided by the terms of Defense Advanced Research Projects Agency (DARPA) Grant No. MDA9720010027 awarded by the Department of Defense.

### FIELD OF THE INVENTION

The present invention relates in general to animal tissue interfaces and, more particularly, to a flexible integrated head-stage as a tissue interface.

### BACKGROUND OF THE INVENTION

Medical research and new product development often involve testing and evaluation of live animal subjects. The live animals are typically mammals, such as rats, mice, rabbits, and monkeys. The testing is necessary to understand the effect and any complication associated with the experimental product or procedure on animals having a similar basic physiology to that of humans, before the product or procedure is approved for human use.

The testing and evaluation may involve blood analysis, tissue analysis, and monitoring of vital organs to observe and record reactions in the test animal to the experimental product or procedure and external stimulus. One of the testing and evaluation techniques involves monitoring and recording neural functions. Many neural functions are electrical in nature. For example, synaptic impulses in the cerebral cortex are essentially electric charges associated with high brain functions such as voluntary movement, sensory information, reactions to stimulus, learning, and memory. The electric charges induced by the synaptic impulses can be recorded with electronic probes or electrodes implanted within the live brain tissue. These neural implants provide electrical signals representative of the brain activities and functions in the test animal.

In the prior art, the electrodes are typically small, rigid micro-wires. The micro-wire electrodes are implanted at selected brain recording sites, for example in the cerebral cortex, and extend up through the skin. The micro-wire electrodes then connect to a head-stage which operates as a neural interface and includes a standard connector for instrument probes and leads. The instrument takes electrical readings from the recording sites.

The process of connecting the head-stage to the implanted micro-wire electrodes is a difficult task, often requiring either sedating the animal or using more than one researcher to perform the task. One person handles the test animal and the other person aligns and makes the connection between the head-stage and the micro-wire electrodes. The process of connecting the head-stage can cause the implanted micro-wire electrodes to move. Moreover, there can be micro-movement in the neural implants just from normal head and body motion of the test animal. The stiff micro-wire electrodes implanted in the brain tissue can cause significant discomfort or anxiety to the test animal, especially during the test procedure. Moreover, the stiff metal structures can cause damage to the surrounding neural or vascular tissues in the brain when the test leads exert a force via the head-stage on the electrodes, or during any relative motion between the brain tissue and the skull. It is important to minimize the discomfort, anxiety, and tissue damage to the test animal which can affect the accuracy and consistency of the test readings.

Another approach is to use polymer-based electrodes which are flexible and absorb some of the movement and torque exerted by outside forces. However, polymer-based electrodes are difficult to implant with any degree of accuracy and consistency because they have little compressive strength, i.e. the electrode tends to bend or buckle when attempting to penetrate the live tissue.

### SUMMARY OF THE INVENTION

In one embodiment, the present invention is a head-stage for implanting as a tissue interface comprising a flexible substrate including a conductor for conducting an electrical signal. A stiffener substrate is coupled to a first end of the flexible substrate. An electronic circuit is supported by the stiffener substrate and has an input coupled to the conductor. An external interface is coupled to an output of the electronic circuit and supported by the stiffener substrate for transmitting the electrical signal.

In another aspect, the present invention is an integrated head-stage comprising an integrated substrate having a first portion forming an electrode for implanting into live tissue and a second portion forming a flexible substrate and including a conductor for conducting an electrical signal. A stiffener substrate is coupled to an end of the flexible substrate opposite the electrode. An external interface is supported by the stiffener substrate for transmitting the electrical signal.

### BRIEF DESCRIPTION OF THE DRAWINGS

FIG. 1 illustrates a test animal with head-stage implant;

FIG. 2 illustrates a cross-sectional view of an electrode and head-stage implanted in the test animal;

FIG. 3 illustrates the electrode for implanting in the test animal;

FIG. 4 illustrates a cross-sectional view of the electrode;

FIGS. 5a-5d illustrate the steps of manufacturing the electrode;

FIG. 6 illustrates an alternate embodiment of the electrode with multiple prongs;

FIG. 7 illustrates the head-stage for implanting in the test animal;

FIG. 8 illustrates a cross-sectional view of the head-stage; and

FIG. 9 illustrates an integrated electrode and head-stage.

### DETAILED DESCRIPTION OF THE DRAWINGS

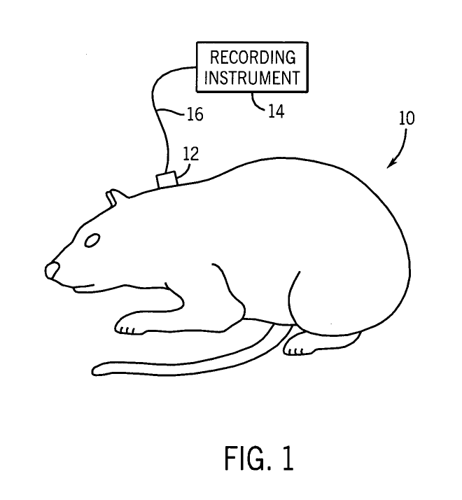

Referring to FIG. 1, test animal 10 is shown. Medical research and new product development often involve testing and evaluation of live animal subjects. The live animals are typically mammals, such as rats, mice, rabbits, and monkeys. The testing is necessary to understand the effect and any complication associated with the experimental product or procedure on animals having a similar basic physiology to that of humans, before the product or procedure is approved for human use. In FIG. 1, test animal 10 is illustrated as a rat.

The testing and evaluation may involve monitoring of vital organs to observe and record reactions in test animal 10 to the experimental product or procedure and external stimulus. In the present description, the brain of test animal 10 is monitored to observe and record neural functions. Many neural functions are reflected in certain patterns of electrical activity. For example, synaptic impulses in the cerebral cortex are essentially electric charges associated with high brain functions such as voluntary movement, sensory information, reactions to stimulus, learning, and memory. The electric charges induced by synaptic impulses can be recorded with electronic probes or electrodes implanted within the live brain tissue. These neural implants provide electrical signals representative of the brain activities and functions in test animal 10.

Test animal 10 is shown with connector 12 extending or extruding through the skin from the back of its neck. Recording instrument 14 is connected by test probes or leads 16 to connector 12. A lab technician or researcher holds test animal 10 in one hand and inserts test leads 16 into connector 12 with the other hand and then locks the test leads in place. The fingers of the hand holding test animal 10, e.g. opposing thumb and index finger, can be used to hold the head steady while test leads 16 are inserted into connector 12. Connector 12 is a zero insertion force (ZIF) type connector. ZIF connector 12 has substantially no resistance to inserting test leads 16 into the connector. Test animal 10 likely experiences minimal sensation to the process of inserting test leads 16 into connector 12, other than the pressure of having its body and head held securely. Since connector 12 extends from the back of the neck of test animal 10, there is less chance of being bitten or receiving undue resistance from the animal. Once the test leads are inserted, a latch or locking mechanism holds test leads 16 secure in connector 12. Recording instrument 14 then monitors and records the signals originating from test animal 10.

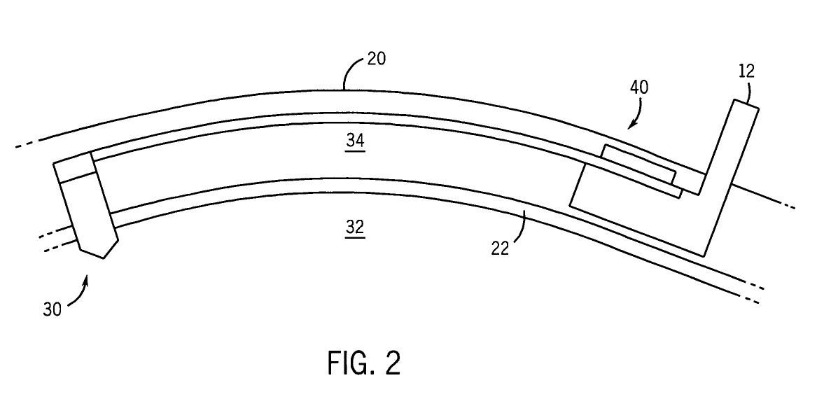

Turning to FIG. 2, a cross-sectional view of the head and neck of test animal 10 is shown. While under general or local anesthesia, skin 20 and a portion of skull or bone structure 22 of test animal 10 are surgically opened. A first end of electrode 30 is implanted or inserted by hand or micromanipulator into live brain tissue 32. Further detail of electrode 30 is provided below. A second end of electrode 30 is connected to connector 38 of head-stage 40. Further detail of head-stage 40 is provided below. Head-stage 40 is positioned in body area 34 between skull 22 and skin 20 from the insertion point of electrode 30 into brain tissue 32 to the exit point on the back of the neck of test animal 10. Head-stage 40 includes a flat flexible portion or substrate which can follow the contour of the body area, e.g. skull 22 and body area 34, and a rigid portion or substrate for supporting external interface components. The flexible portion of head-stage 40 provides freedom of movement to reduce discomfort to test animal 10. Connector 12 is an external interface component of head-stage 40. Connector 12 exits through skin 20 on the back of the neck of test animal 10 to connect to test leads 16 and recording instrument 14.

Electrode 30 and head-stage 40 shown in the figures is not necessarily drawn to scale for purposes of illustration and may differ in relative proportions in practice. In the figures, common reference numerals are used for elements which provide the same or similar function.

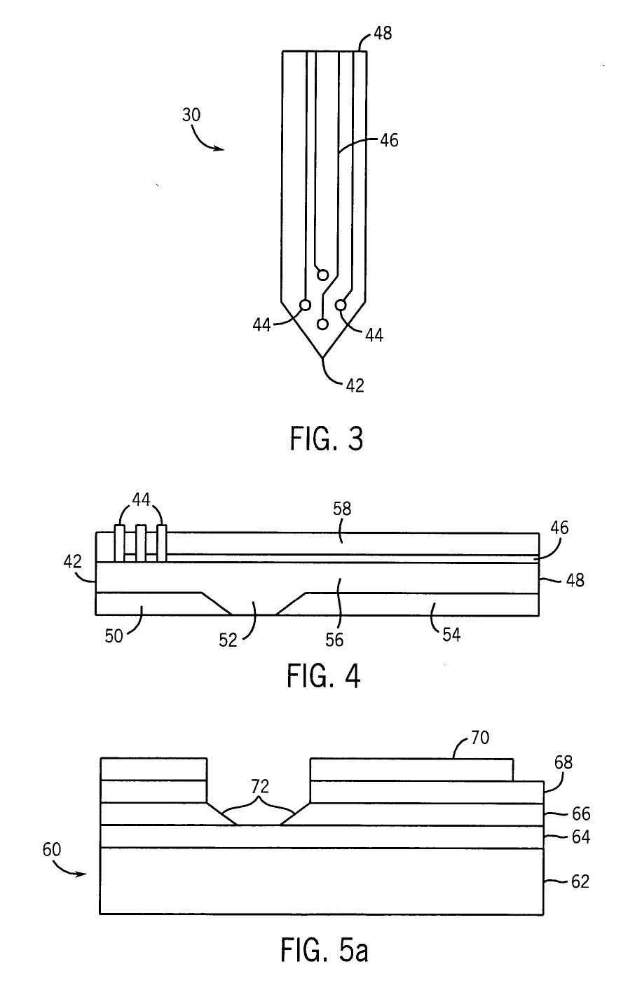

Further detail of electrode 30 is shown in FIG. 3. Electrode 30 is a polymer-based micro-electromechanical system (MEMS) suitable for use as a small, strong, and moisture repellent neural implant. Electrode 30 is designed to reduce damage when inserted into brain tissue 32 of test animal 10. Electrode 30 has a pointed end 42 for easy and positive penetration into brain tissue 32. Pointed end 42 includes a plurality of recording sites 44, which when electrode 30 is implanted, come in physical contact with certain areas of brain tissue 32. Recording sites 44 receive electric charges or action potential from the areas of brain tissue 32 which are intended to be monitored. In response to stimulus or physical activity, the neural functions in the brain cause changes in local field potential which are picked up by recording sites 44. The electric charges and action potential incident to each recording site 44 become or are converted to electrical signals which are transmitted along conductors 46 to connector end 48 of electrode 30. Conductors 46 may run along the surface of electrode 30 as shown, or be routed through intermediate layers of electrode 30. Recording sites 44 and conductors 46 are made with gold traces. Conductors 46 connect to connector 38 of head-stage 40 to route the electrical signals from recording sites 44 to head-stage 40. Electrode 30 has an impedance range from 700 kilo-ohm to 1 mega-ohm at 1 kilo-Hertz for signal gain and high signal to noise ratio.

In another embodiment, recording sites 44 include transducers to covert physical phenomenon such as pressure, temperature, sound, optical, and chemical reactions into electrical signals. Electrode 30 with transducers on recording sites 44 can be used to monitor a variety of body functions and can be located in other parts of the body, e.g. muscles, lungs, heart, gastro-intestinal organs, and spinal column. Again, the electrical signals are routed from recording sites 44 to head-stage 40.

A cross-sectional view of electrode 30 is shown in FIG. 4. A silicon substrate 50 forms a rigid backbone for electrode 30. Substrate 50 is between 2-10 micrometers (μm) in thickness, and about 0.2 millimeters (mm) in width and 1.5 to 2.0 mm from the tip of pointed end 42 to the start of flexible portion 52. Substrate 50 provides a rigid structure and compressive strength for ease of penetration of electrode 30 into brain tissue 32. Electrode 30 is inserted into brain tissue 32 of test animal 10 approximately 1.5 to 2.0 mm. Silicon substrate portion 54 extends from flexible portion 52 to connector end 48 to provide another portion of the rigid backbone and additional rigidity and compressive strength for electrode 30.

Electrode 30 has an intermediate polymer layer 56 disposed on substrates 50 and 54. Polymer layer 56 is made of benzocyclobutene (BCB) or polyimide material. BCB is suitable for electrode 30 because its flexibility, biocompability, a high degree of planarization, and low dielectric constant. Flexible portion 52 is an extension of polymer layer 56 disposed between substrates 50 and 54. Flexible portion 52 is about 1.0 mm in length. Flexible portion 52 is beveled or angled with substrates 50 and 54. Given that the portion of electrode 30 from the tip of pointed end 42 to the start of flexible portion 52 is implanted in brain tissue 32, then flexible portion 52 itself is positioned in a space between brain tissue 32 and skull 22.

Flexible portion 52 provides flexibility and absorbs stress from any relative movement brain tissue 32 and outside forces. In the event of any motion in head-stage 40 or movement in connector end 48 of electrode 30, or given any micro-movement between skull 22 and brain tissue 32, then the portion of electrode 30, e.g. from the tip of pointed end 42 to the start of flexible portion 52, remains substantially fixed in position relative to brain tissue 32. The portion of electrode 30 from flexible portion 52 to connector end 48 moves with the outside forces. In part, flexible portion 52 provides for the isolation and independent movement in the different portions of electrode 30. Since the implanted portion of electrode 30 does not move relative to brain tissue 32, then test animal 10 does not experience discomfort or damage to the live tissue. The test readings are more accurate and consistent.

Conductors 46 may be routed along intermediate polymer layer 56 between recording sites 44 and connector end 48 of electrode 30. A top polymer layer 58 is disposed over intermediate polymer layer 56 to provide additional flexibility and encapsulate conductors 46. Polymer layer 58 is also made of BCB or polyimide material. As shown in FIG. 3, conductors 46 may be routed along the top surface of polymer layer 58.

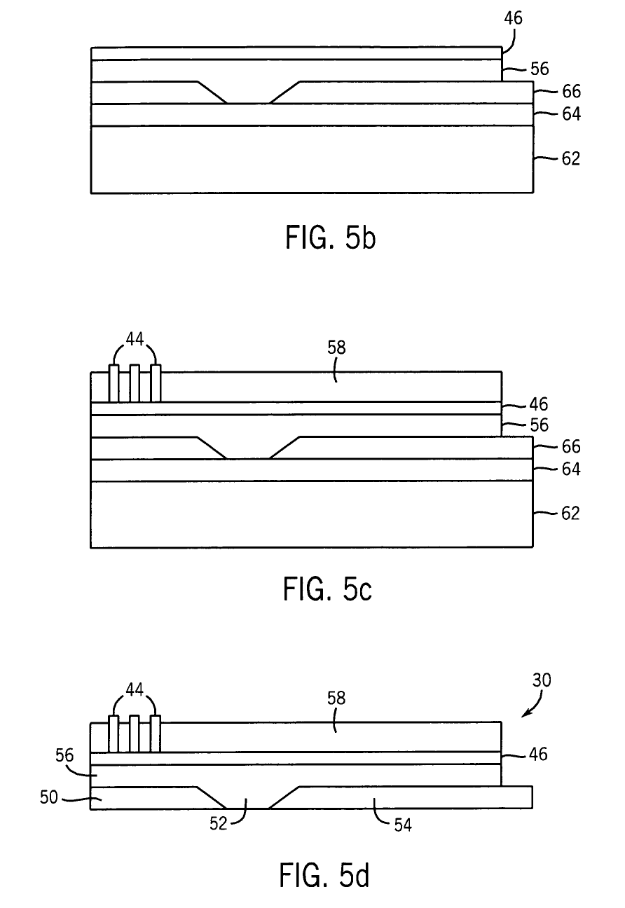

The manufacturing process of electrode 30 is shown in FIGS. 5a-5d. In FIG. 5a, silicon-on-insulator (SOI) substrate 60 is provided. SOI substrate 60 includes silicon layer 62, silicon dioxide layer 64, and silicon layer 66. A metal layer 68 is disposed on silicon layer 66. Metal layer 68 may include gold, nickel, and copper. A photoresist layer 70 is applied to metal layer 68 and patterned and developed. A portion of metal layer 68 is etched away using reactive ion etching (RIE). A portion of silicon layer 66 is then wet etched using 7% Tetra Methyl Ammonium Hydroxide (TMAH) solution. The silicon-etching rate depends on the crystal planes in TMAH. The (100) crystal plane has a much faster etch rate than the (111) plane. The difference in etch rate forms a beveled or angled surfaces 72.

In FIG. 5b, metal layer 68 and photoresist layer 70 are removed to expose silicon layer 66 with beveled edges 72. A first layer of BCB or polyimide material is spin-coated, exposed, and then developed to form intermediate polymer layer 56. The BCB fills in the area between beveled edges 72 as well as forming polymer layer 56. BCB generally requires less cure time than polyimide material. A gold layer is deposited on polymer layer 56 using an electron beam evaporation chamber to form conductors 46.

In FIG. 5c, a second layer of BCB or polyimide material is spun, exposed, and developed to form polymer layer 58 and encapsulate conductors 46. Openings are formed in polymer layer 58 for recording sites 44.

In FIG. 5d, silicon layer 62 is removed by RIE. Silicon dioxide layer 64 is dissolving in 49% hydrofluoric (HF) acid solution. The resulting structure comprises electrode 30.

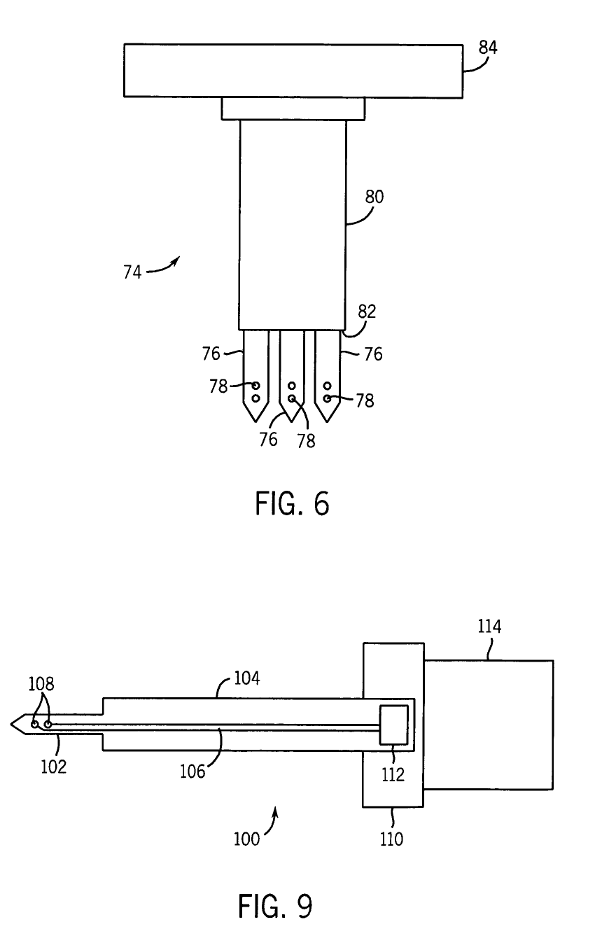

An alternate embodiment of the implant electrode is shown in FIG. 6. Electrode 74 includes multiple prongs 76 with each prong 76 having multiple recording sites 78. Prongs 76 and electrode body or shaft 80 are constructed as described for electrode 30 with first and second polymer layers for flexibility and a rigid silicon backbone layer for stiffness and compressive strength when inserting electrode 74 into live tissue. Electrode body 80 further includes a flexible portion like 52 above shank 82 to provide a freedom of movement of body 80 with respect to prongs 76. Again, prongs 76 implanted in brain tissue 32 remain substantially fixed in the event of outside forces. The flexible portion like 52 and polymer layers isolate any movement in the electrode external to brain tissue 32. Shank 82 also acts as a stop for prongs 76 to set electrode 74 the correct depth into the live tissue. A plurality of conductors are routed from recording sites 78 along body 80 to connector 84 for connection to head-stage 40.

As described above, electrode 30 has features of rigid mechanical stiffness, as provided by substrates 50 and 54, and flexibility, as provided by flexible portion 52 and polymer layers 56 and 58. The mechanical stiffness makes for ease of penetration of electrode 30 into brain tissue 32. The flexibility of electrode 30 reduces or prevents damage to neural or vascular tissues in the brain in and around electrode 30. If the event of any relative motion between skull 22 and brain tissue 32 of test animal 10, or any motion of head-stage 40 from external forces, the portion of electrode 30 implanted in brain tissue 32, i.e. between flexible portion 52 and pointed end 42, remains substantially fixed relative to brain tissue 32. The portion of electrode 30 from flexible portion 52 to connector end 48 moves with skull 22 and/or head-stage 40. In other words, flexible portion 52 accommodates and allows for micro-movement between skull 22 and brain tissue 32, or movement between head-stage 40 and brain tissue 32. Connector end 48 of electrode 30 moves with the outside forces while the implanted portion of electrode 30 is held substantially motionless relative to brain tissue 32. The flexible portion 52 and polymer layer 56 and 58 provide the isolation of end 42 from outside forces to reduce discomfort to test animal 10 and damage to brain tissue 32. With less discomfort, trauma, and anxiety to test animal 10, the intended behavior or activity can be more accurately observed and recorded.

Electrode 30 is useful in human and animal subjects where it is desirable to have a rigid structure for accurate and consistent insertion of the electrode into the tissue to be monitored. With transducers on recording sites 44, electrode 30 is useful in monitoring and recording a variety of physical phenomenon which can be converted to electrical signals and transmitted along conductors 46. Electrode 30 can be placed in many different body areas of the subject to monitor and record bodily functions. For example, electrode 30 can be used to monitor internal organs and muscular activity.

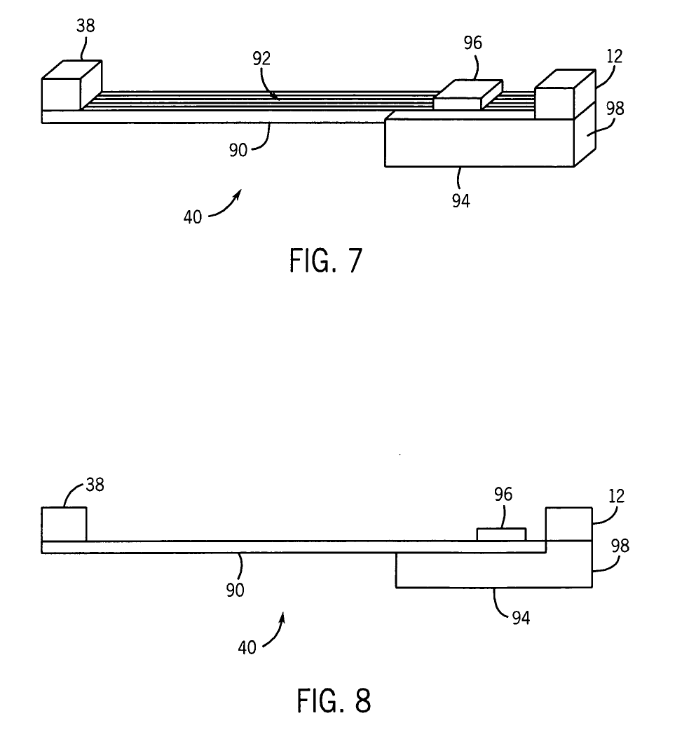

Further detail of head-stage 40 is shown in FIG. 7. Head-stage 40 includes connector 38 for connecting to electrode 30 with minimal force. Connector 38 can be a ZIF type connector. Flexible substrate 90 connects to conductor 38 and includes a plurality of conductors 92 for transmitting the electrical signals received from recording sites 44 on electrode 30. Substrate 90 is a flat ribbon made of BCB, polyimide, or other suitable polymer material to provide strength and flexibility. Substrate 90 may be up to 60 cm or more in length. Conductors 92 may be formed on both sides of substrate 90 to increase the number of conductors and correspondingly the number of recording sites 44 on electrode 30.

Head-stage 40 further includes stiffener portion or substrate 94. Stiffener portion 94 is a rigid substrate about 2 centimeters (cm) by 2 cm and supports a portion of flexible substrate 90. Stiffener portion 94 is made from silicon. Alternatively, conductors 92 of flexible substrate 90 connect to conductors on stiffener portion 94. An electronic circuit 96 is provided on the portion of substrate 90 supported indirectly by stiffener portion 94, or disposed directly on stiffener portion 94 itself. Electronic circuit 96 is a CMOS integrated circuit and operates as part of the external interface to perform signal conditioning and signal processing functions for the electrical signals. For example, electronic circuit 96 may provide buffering, amplification, and filtering for the electrical signals. Electronic circuit 96 includes necessary programming and control logic to perform the signal processing. In addition, electronic circuit 96 may multiplex the electronic signals to fewer conductors on its output. Multiplexing allows for more recording sites 44 without increasing the number of output leads for connector 12. In fact, by multiplexing the electrical signals, connector 12 needs only one signal conductor in a minimal configuration.

Electronic circuit 96 may receive operating potential from recording instrument 14 by way of test leads 16. Alternatively, a power source or battery pack is disposed within stiffener portion 94 to provide operating potential to electronic circuit 96. Electronic circuit 96 may be coupled to a wireless transmitter, e.g. radio frequency (RF) transmitter, which operates as an external interface to transmit electrical signals to recording instrument 14. If electronic circuit 96 uses a wireless transmitter, connector 12 and the corresponding exit point from the back of the neck of test animal 10 can be eliminated, which negates a point of irritation and infection for test animal 10. In another embodiment of the external interface, electronic circuit 96 may convert the electrical signals to optical patterns for transmission along fiber-optic cables, or by infrared transmission, to recording instrument 14.

Connector 12 is mounted on the leading edge of stiffener portion 94 for a zero degree angle on insertion. Connector 12 is a ZIF type connector for less traumatic connection of test leads 16 to head-stage 40. In other embodiments, connector 12 is rotated 90 degrees to side 98 of stiffener portion 94 for a bottom-up or other orientation insertion.

The electrical signals from recording sites 44 on electrode 30 are routed to connector 38, along conductors 92 to electronic circuit 96. Electronic circuit 96 performs signal processing and conditioning on the electrical signals and sends the conditioned electrical signals by way of connector 12 and test leads 16 to recording instrument 14 for monitoring and recording.

In addition to transmitting electrical signals from recording sites 44 on electrode 30 to connector 12 and recording instrument 14, electronic circuit 96 and conductors 92 on head-stage 40 can also transmit electrical signals to recording sites 44. The electrical signals sent to recording sites 44 may be used to program or calibrate the transducers. In addition, the electrical signals could be used to stimulate the tissue in which electrode 30 is implanted.

The combination of flexible substrate 90 and stiffener portion 94 offers a number of useful advantages. Substrate 90 is lightweight and flexible which reduces any discomfort and anxiety experienced by test animal 10. Reducing the invasiveness of the test implants and testing procedure allows for observation and recordation of the intended behavior or activity in the test subject, which is helpful in taking accurate measurements of neural activity. The flexibility of substrate 90 provides for ease of implant and adaptability to follow the contour of the body area. Stiffener portion 94 provides a rigid support for electronic circuit 96 and connector 12. Stiffener portion 94 also provides a solid base to simplify the insertion of test lead 16 into connector 12. Furthermore, by locating electronic circuit 96 and the exit point in skin 20 for connector 12 some distance from electrode 30, test animal 10 is less subject to infection, at least in the dangerous area where brain tissue 32 has been exposed by the surgical implantation procedure.

In FIG. 9, an integrated electrode and head-stage 100 is shown. The integrated electrode and head-stage 100 removes the need for connector 38. Electrode 102 is constructed similar to electrode 30 with first and second polymer layers, rigid silicon backbone like 50 and 54, and flexible portion like 52. Electrode 102 is integrated with flexible substrate 104. That is, electrode 102 and flexible substrate 104 are made from the same process and same material to form one continuous substrate. The integrated electrode and substrate is flexible to allow electrode 102 to bend up to 90 degrees for insertion into the test animal. The flexible portion like 52 allows tip of electrode 102, which is implanted in the brain tissue, freedom of movement with respect to the remaining portion of electrode 102. Flexible substrate 104 contours to the body area. Conductors 106 are routed from recording sites 108 along substrate 104 to stiffener portion 110. Electronic circuit 112 is disposed substrate 104 and supported by stiffener portion 110. Electronic circuit 112 performs signal processing on the electrical signals from recording sites 108. The electrical signals are sent to recording instrument 14 by way of connector 114.

A person skilled in the art will recognize that changes can be made in form and detail, and equivalents may be substituted, for elements of the invention without departing from the scope and spirit of the invention. The present description is therefore considered in all respects to be illustrative and not restrictive, the scope of the invention being determined by the following claims and their equivalents as supported by the above disclosure and drawings.

1. A head-stage for implanting as a tissue interface, comprising:
a first connector coupled for receiving a plurality of electrical signals;
a flexible substrate coupled to the first connector and including a plurality of conductors for the electrical signals;
a stiffener substrate coupled to a portion of the flexible substrate;
an electronic circuit disposed on the flexible substrate above the stiffener substrate and having inputs coupled to the plurality of conductors; and
a second connector supported by the stiffener substrate and coupled to an output of the electronic circuit.

2. The head-stage of claim 1 wherein the flexible substrate includes benzocyclobutene.

3. The head-stage of claim 1 wherein the flexible substrate includes polyimide.

4. The head-stage of claim 1 wherein the flexible substrate overlies a portion of the stiffener substrate.

5. The head-stage of claim 1 wherein the electronic circuit performs signal processing on the electrical signals.

6. The head-stage of claim 1 wherein the flexible substrate and stiffener substrate are implanted under a skin surface of a test subject.

7. The head-stage of claim 1 wherein the second connector is a zero insertion force type connector.

8. A head-stage, comprising:
a flexible substrate including a conductor for conducting an electrical signal;
a stiffener substrate coupled to a first end of the flexible substrate;
an electronic circuit supported by the stiffener substrate and having an input coupled to the conductor; and
an external interface coupled to an output of the electronic circuit and supported by the stiffener substrate for transmitting the electrical signal.

9. The head-stage of claim 8 wherein the flexible substrate includes benzocyclobutene.

10. The head-stage of claim 8 wherein the external interface includes a first connector supported by the stiffener substrate and coupled to an output of the electronic circuit.

11. The head-stage of claim 10 wherein the first connector is a zero insertion force type connector.

12. The head-stage of claim 10 further including a second connector coupled to a second end of the flexible substrate.

13. The head-stage of claim 8 wherein the flexible substrate overlies a portion of the stiffener substrate.

14. The head-stage of claim 8 wherein the flexible substrate and stiffener portion are implanted under a skin surface of a test subject.

15. The head-stage of claim 8 wherein the electronic circuit conducts the electrical signal bi-directionally along the conductor.

16. An integrated head-stage, comprising:
an integrated substrate having a first portion forming an electrode for implanting into live tissue and a second portion forming a flexible substrate and including a conductor for conducting an electrical signal;
a stiffener substrate coupled to an end of the flexible substrate opposite the electrode; and
an external interface supported by the stiffener substrate for transmitting the electrical signal.

17. The integrated head-stage of claim 16 wherein the external interface includes an electronic circuit disposed above the stiffener substrate and having an input coupled to the conductor.

18. The integrated head-stage of claim 17 wherein the external interface further includes a first connector supported by the stiffener substrate and coupled to an output of the electronic circuit.

19. The integrated head-stage of claim 18 wherein the first connector is a zero insertion force type connector.

20. The integrated head-stage of claim 16 wherein the electrode and flexible substrate include benzocyclobutene.

21. The integrated head-stage of claim 16 wherein the flexible substrate overlies a portion of the stiffener substrate.

22. A head-stage for implanting as a tissue interface, comprising:
a flexible substrate including a conductor for conducting an electrical signal;
a stiffener substrate coupled to the flexible substrate; and
an external interface supported by the stiffener substrate for transmitting the electrical signal.

23. The head-stage of claim 22 wherein the flexible substrate includes benzocyclobutene.

24. The head-stage of claim 22 wherein the external interface includes an electronic circuit disposed above the stiffener substrate and having an input coupled to the conductor.

25. The head-stage of claim 24 wherein the external interface further includes a first connector supported by the stiffener substrate and coupled to an output of the electronic circuit.

26. The head-stage of claim 25 wherein the first connector is a zero insertion force type connector.

27. The head-stage of claim 22 wherein the electronic circuit conducts the electrical signal bi-directionally along the conductor.
